(19) United States
(12) Patent Application Publication
(10) Pub. No.: US 2025/0011831 A1
(43) Pub. Date: Jan. 9, 2025

(54) Method for Verifying the Result of a Process of Cleaning Simulated Chemical and/or Microbiological Contamination Applied Beforehand to a Test Area

(71) Applicant: Skan AG, Allschwil (CH)

(72) Inventors: Gregor Hommes, Wachtendonk (DE); Davide Ravasio, Wangen bei Olten (CH)

(73) Assignee: Skan AG, Allschwil (CH)

(21) Appl. No.: 18/711,004

(22) PCT Filed: Nov. 18, 2022
(86) PCT No.: PCT/CH2022/000009

§ 371 Date: May 16, 2024

(30) Foreign Application Priority Data
Nov. 19, 2021 (EP) 21405006.4

(51) Int. Cl. C12Q 1/22

(52) U.S. Cl. CPC C12Q 1/22

(57) ABSTRACT

The invention relates to a method for verifying the result of a process of cleaning simulated chemical and/or microbiological contamination applied beforehand to a test area, in particular for the validation process for automated washing system functions. The method comprises the following steps: —providing a coating device having a filled cartridge, which contains a test liquid for simulating chemical and/or microbiological contamination. The test liquid contains a defined type and concentration of chemically and/or microbiologically contaminating substances; —selecting a defined test area and applying a defined amount of test liquid to the test area; —carrying out a standardised process of cleaning the test area provided with the test liquid; and—finally verifying and logging the result of the cleaning process carried out.

### FIELD OF APPLICATION OF THE INVENTION

The present invention relates to a method for verifying the result of a cleaning process to remove simulated chemical and/or microbiological contaminants previously applied to a test area. The invention relates in particular to the validation process for automated wash-system functions of the “Cleaning in Place” (CIP) and “Washing in Place” (WIP) types.

### PRIOR ART

U.S. Pat. No. 3,841,973 discloses a mechanical wiping apparatus for automated microbiological sampling of a surface. A motor-driven arm supports a swab which travels over the surface to be tested in a defined pattern of movement and with a defined contact pressure, and the swab contaminated as a result is subsequently analyzed. A similar device for automated cleaning validation for a surface to be checked is described in WO 2014/089 320 A1. Residual contaminants still remaining on the surface after the cleaning process are picked up by a swab moved on the surface and are quantified by analysis.

U.S. Pat. No. 6,789,965 B2 relates to a device and a method for applying biological or chemical substances via capillary tubes in a high-resolution and defined formation to a test plate for diagnostic purposes, for example for DNA analyses.

The flyer “Cleanability of Surfaces—CSM Riboflavin Test” from the Fraunhofer Institute for Manufacturing Engineering and Automation IPA [see: https://www.ipa.fraunhofer.de/content/dam/ipa/en/documents/Expertises/Reinst-und-Mikroproduktion/Flyer_Cleanability_of_Surfaces.pdf (fraunhofer.de) Flyer_Cleanability_of_Surfaces.pdf (fraunhofer.de)] also deals with the present issue. Described is a test method for checking the effectiveness of complete cleaning/washing cycles on surfaces by using a test liquid containing the water-soluble and highly light-sensitive B2 vitamin riboflavin.

For a test, the surfaces of the relevant equipment that are to be cleaned are first sprayed with a water-riboflavin solution. These surfaces are then sprayed with a rinse solution according to the method of a timed burst rinse, followed by a timed gravity drain, preferably three times. After completion of the rinse phase, highly critical surface areas in particular are visually inspected using a black light and telescoping mirror in order to determine whether the riboflavin solution has been completely removed. Complete, proper cleaning is confirmed by the absence of visible fluorescence. However, if there are residues showing under the black light, the technician will identify insufficiently cleaned surface areas and have to adjust the intensity and/or aim of the spray device

### OBJECT OF THE INVENTION

Although the riboflavin test provides a certain qualitative check of the thoroughness of the cleaning performed, it does not provide quantitative proof of whether the surfaces treated in the cleaning process are actually completely free of highly potent or sensitizing substances, or are only contaminated with extremely low levels of residues, i.e., levels that are still acceptable, in order to avoid hygiene risks or cross-contamination risks, which is essential for qualified validation.

Validation of proper cleaning (Performance Qualification-PQ) of equipment surfaces and employed functional parts to be treated in the pharmaceutical industry in particular by means of washing devices is ultimately the responsibility of the users, but the users of washing devices do expect the manufacturers to also provide validation methods for proof of a highly challenging level of cleanliness. It is therefore an object of the present invention to propose a validation method for the relevant field of application that is efficient in its use and highly reliable and sensitive.

### OVERVIEW OF THE INVENTION

The method according to the invention is designed for verification of the result of a cleaning process to remove simulated chemical and/or microbiological contaminants previously applied to a test area and comprises the following steps:
first providing a coating device having a filled cartridge containing a test liquid for simulation of chemical and/or microbiological contaminants, wherein the test liquid contains a defined type and concentration of chemically and/or microbiologically contaminating substances;
then selecting a defined test area and applying a defined quantity of test liquid to the test area;
additionally performing a standardized cleaning process on the test area provided with the test liquid; and
lastly verifying and logging the result of the cleaning process performed.

Defined hereinbelow are specific embodiments of the invention:

After the application of the defined quantity of test liquid to the test area and before the performance of the standardized cleaning process on the test area provided with the test liquid, the intensity and possibly also the type of the contaminant present on the test area is determined and logged.

The test area is defined on the surface of an installation, for example a containment for production or research purposes in relation to pharmaceutical or biotechnological products or in medical technology. The test area can also be defined on the surface of a functional part usable in the installation.

Alternatively, the test area used is a separate piece of material in the form of an indicator plate which is identical in terms of its material characteristics to the material used in the construction of the installation or to the material used for the functional part, which is acceptable as reference material in any case, and which can for example also be in the form of a film.

The coating device to be provided for the cartridge for simulation of chemical and/or microbiological contaminants and filled with the test liquid is of the inkjet printer type. In one refinement, the coating device is adjustable to different coating quantities and coating patterns and/or to test areas of different dimensions and preferably has the form of a handheld device.

The contaminating substance to be added to the test liquid are particles and/or a solvate. The test liquid mainly consists of a neutral solvent and, added thereto, chemical and/or microbiological substances, such as viruses, fungi, spores, bacteria, proteins or cellular fragments, in a defined concentration for simulation of contaminants on the test area.

Alternatively, the test liquid again mainly consists of a neutral solvent and chemical substances present in the solvent and/or a two-component or multicomponent mixture of microbiological substances from the group consisting of viruses, fungi, spores, bacteria, proteins and cellular fragments, in a defined concentration for simulation of contaminants on the test area.

The verification of the result of the cleaning process performed when using a first-form test area comprises the following steps:
wiping over the previously contaminated and subsequently cleaned surface of the test area with a neutral cleaning element;
removing the traces picked up from the surface of the test area by the cleaning element on wiping, using a solvent; and
testing the traces-loaded solvent in an analyzer; wherein
the analysis result indicates whether the cleaning process was successful or whether residues of the contaminating test liquid above the permissible level remained on the test area, meaning that the cleaning process must be repeated or be intensified from the beginning in order to achieve an acceptable level of cleanliness.

The verification of the result of the cleaning process performed when using a second-form test area comprises the following steps:
treating the previously contaminated and subsequently cleaned surface of the second-form test area in a vessel containing a solvent; and
testing the traces-loaded solvent in an analyzer; wherein
the analysis result indicates whether the cleaning process was successful or whether residues of the contaminating test liquid above the permissible level remained on the test area, meaning that the cleaning process must be repeated or be intensified from the beginning in order to achieve an acceptable level of cleanliness.

Before the application of the test liquid to the indicator plate selected as test area by means of the coating device, a positioning device is used for exact alignment of the indicator plate under the printer head of the coating device.

### EXEMPLARY EMBODIMENT

The method provided for verification of the result of a cleaning process executed especially by a washing device to remove simulated chemical and/or microbiological contaminants previously applied to a test area comprises the sequence of the following steps:

### Step 1

Providing a coating device having a filled cartridge containing a test liquid for simulation of chemical and/or microbiological contaminants, wherein the test liquid contains a defined type and concentration of chemically and/or microbiologically contaminating substances. The contaminating substance to be added to the test liquid are particles and/or a solvate. The test liquid mainly consists of a neutral solvent and, added thereto, chemical and/or microbiological substances, such as viruses, fungi, spores, bacteria, proteins or cellular fragments, in a defined concentration for simulation of contaminants on the test area. Alternatively, the test liquid again mainly consists of a neutral solvent and chemical substances present in the solvent and/or a two-component or multicomponent mixture of microbiological substances from the group consisting of viruses, fungi, spores, bacteria, proteins and cellular fragments, in a defined concentration for simulation of contaminants on the test area.

The coating device to be provided for the cartridge for simulation of chemical and/or microbiological contaminants and filled with the test liquid is of the inkjet printer type. In one refinement, the coating device is adjustable to different coating quantities and coating patterns and/or to test areas of different dimensions and preferably has the form of a handheld device.

### Step 2

Selecting a defined test area and applying a defined quantity of test liquid to the test area. The test area is defined on the surface of an installation, for example a containment for production or research purposes in relation to pharmaceutical or biotechnological products or in medical technology. The test area can also be defined on the surface of a functional part usable in the installation. Alternatively, the test area used is a separate piece of material which is identical in terms of its material characteristics to the material used in the construction of the installation or to the material used for the functional part, which is acceptable as reference material in any case. Two different forms of the test area can thus be defined. The first form is a real surface section of the installation or of a functional part usable therein. By contrast, the second form is an indicator plate, either consisting of a single piece of material identical to the real surface section or consisting of an adequate reference material. After the application of the defined quantity of test liquid to the first-form or second-form test area and before the performance of the standardized cleaning process on the test area provided with the test liquid, the intensity and possibly also the type of the contaminant present on the test area is determined and logged. The application of the test liquid to the test area is preferably effected by spraying, in particular printing.

### Step 3

Performing a standardized cleaning process on the first-form or second-form test area provided with the test liquid. Such a cleaning process is effected by spraying using a rinse solution, preferably with control of time and intensity and, if necessary, in multiple cycles. Where appropriate, the rinse solution is discharged by gravity drain or by suction.

### Step 4

The preceding steps are followed by verification and logging of the result of the cleaning process performed. The entire process flow can now be summarized as follows, a differentiation being made according to whether a first-form test area or a second-form test area is selected. Thus, according to step 2, a real surface section of the installation or of a functional part usable therein is defined as a first-form test area or an indicator plate is defined as a second-form test area.

Process Flow with First-Form Test Area (Real Surface Section)

By means of the coating device according to step 1, a defined quantity of test liquid is applied to the test area. This is followed by, according to step 3, the performance of a standardized cleaning process on the test area provided with the test liquid.

Lastly, step 4 is required to check whether the result of the cleaning process performed is acceptable as per regulations. To this end, the surface of the test area that was first contaminated and then cleaned in step 3 is wiped over with a neutral cleaning element. Thereafter, the traces picked up from the surface of the test area by the cleaning element on wiping are removed using a solvent and tested in an analyzer. The analysis result indicates whether the cleaning process was successful or whether residues of the contaminating test liquid above the permissible level remained on the test area, meaning that the cleaning process must be repeated or be intensified from the beginning in order to achieve an acceptable level of cleanliness.

Process Flow with Second-Form Test Area (Indicator Plate)

According to good practice, the test area used is an indicator plate—the technical term being “coupon”—which is in line with the above characterization. Before the application of the test liquid to the indicator plate by means of the coating device according to step 1, the indicator plate is inserted into a positioning device for exact alignment of said indicator plate under the printer head of the coating device. Once again, a defined quantity of test liquid is, then, applied, followed by, according to step 3, the performance of a standardized cleaning process on the indicator plate provided with the test liquid. Preferably, the indicator plate remains in the positioning device during this operation.

Finally, a check must be carried out in step 4 to determine whether the cleaning process performed yielded a perfect result. To this end, the indicator plate which was first specifically contaminated and then cleaned in step 3 is treated in a vessel containing solvent. The residues removed thereby are tested in an analyzer. The analysis result leads to the finding of whether the cleaning process was sufficiently effective or whether residues from the contaminating test liquid remained on the indicator plate to an unacceptable extent, meaning that the cleaning must be repeated or be intensified from the beginning in order to achieve the required level of cleanliness.

1. A method for verifying the result of a cleaning process to remove simulated chemical and/or microbiological contaminants previously applied to a test area, comprising the following steps:
a) providing a coating device having a filled cartridge containing a test liquid for simulation of chemical and/or microbiological contaminants, wherein the test liquid contains a defined type and concentration of chemically and/or microbiologically contaminating substances;
b) selecting a defined test area and applying a defined quantity of test liquid to the test area by means of the coating device;
c) performing a standardized cleaning process on the test area provided with the test liquid; and
d) verifying and logging the result of the cleaning process performed.

2. The method as claimed in claim 1, wherein after the application of the defined quantity of test liquid to the test area and before the performance of the standardized cleaning process on the test area provided with the test liquid, the intensity and possibly also the type of the contaminant present on the test area is determined and logged.

3. The method as claimed in claim 1, wherein the test area in a first form is defined on the surface:
a) of an installation, for example a containment for production or research purposes in relation to pharmaceutical or biotechnological products or in medical technology; or
b) of a functional part usable in the installation.

4. The method as claimed in claim 1, wherein the test area used in an alternative second form is a separate piece of material in the form of an indicator plate which is identical in terms of its material characteristics to the material used in the construction of the installation or to the material used for the functional part or which is acceptable as reference material in any case.

5. The method as claimed in claim 1, wherein the coating device is of the inkjet printer type.

6. The method as claimed in claim 5, wherein the coating device is adjustable to different coating quantities and coating patterns.

7. The method as claimed in claim 6, wherein the coating device is adjustable to test areas of different dimensions.

8. The method as claimed in claim 7, wherein the coating device is designed as a handheld device.

9. The method as claimed in claim 1, wherein the contaminating substance to be added to the test liquid are particles and/or a solvate.

10. The method as claimed in claim 9, wherein the test liquid consists of:
a) a neutral solvent; and
b) chemical and/or microbiological substances present in the solvent, such as viruses, fungi, spores, bacteria, proteins or cellular fragments, in a defined concentration for simulation of contaminants on the test area.

11. The method as claimed in claim 10, wherein the test liquid consists of:
a) a neutral solvent; and
b) chemical substances present in the solvent and/or a two-component or multicomponent mixture of microbiological substances from the group consisting of viruses, fungi, spores, bacteria, proteins and cellular fragments, in a defined concentration for simulation of contaminants on the test area.

12. The method as claimed in claim 1, wherein the verification of the result of the cleaning process performed comprises the following steps:
a) wiping over the previously contaminated and subsequently cleaned surface of the first-form test area with a neutral cleaning element;
b) removing the traces picked up from the surface of the test area by the cleaning element on wiping, using a solvent; and
c) testing the traces-loaded solvent in an analyzer; wherein
d) the analysis result indicates whether the cleaning process was successful or whether residues of the contaminating test liquid above the permissible level remained on the test area, meaning that the cleaning process must be repeated or be intensified from the beginning in order to achieve an acceptable level of cleanliness.

13. The method as claimed in claim 1, wherein for verification of the result of the cleaning process performed comprises the following steps:
a) treating the previously contaminated and subsequently cleaned surface of the second-form test area in a vessel containing a solvent; and
b) testing the traces-loaded solvent in an analyzer; wherein
c) the analysis result indicates whether the cleaning process was successful or whether residues of the contaminating test liquid above the permissible level remained on the test area, meaning that the cleaning process must be repeated or be intensified from the beginning in order to achieve an acceptable level of cleanliness.

14. The method as claimed in claim 1, wherein before the application of the test liquid to the indicator plate selected as test area by means of the coating device, a positioning device is used for exact alignment of the indicator plate under the printer head of the coating device.
